(19) United States
(12) Patent Application Publication
(10) Pub. No.: US 2021/0299437 A1
(43) Pub. Date: Sep. 30, 2021

(54) FLEXIBLE AND SELF-BONDING IMPLANTABLE ELECTROSTIMULATION DEVICE

(71) Applicant: The Board of Trustees of the Leland Stanford Junior University, Stanford, CA (US)

(72) Inventors: Yuxin Liu, Stanford, CA (US); Jinxing Li, Stanford, CA (US); Zhenan Bao, Stanford, CA (US)

(73) Assignee: The Board of Trustees of the Leland Stanford Junior University, Stanford, CA (US)

(21) Appl. No.: 17/196,477

(22) Filed: Mar. 9, 2021

Related U.S. Application Data
(60) Provisional application No. 63/002,950, filed on Mar. 31, 2020.

(51) Int. Cl. A61N 1/05

(52) U.S. Cl. CPC A61N 1/0558

(57) ABSTRACT

Example implementations include an implantable device with a first substantially planar panel having a first planar surface and a second planar surface opposite to the first planar surface, at least one electrode portion at the first planar surface, and a panel elasticity corresponding to a tissue elasticity associated with an in vivo nerve, a second substantially planar panel having a first planar surface adhered to the second planar surface of the first panel, at least one sensor portion disposed on the first planar surface of the second panel, and the panel elasticity, and a third substantially planar panel having a first planar surface adhered to the first planar surface of the first panel, and the panel elasticity.

### CROSS-REFERENCE TO RELATED PATENT APPLICATIONS

This application claims priority to U.S. Provisional Patent Application Ser. No. 63/002,950, entitled “MORPHING ELECTRONICS THAT ENABLE NEUROMODULATION IN GROWING TISSUE,” filed Mar. 31, 2020, the contents of all such applications being hereby incorporated by reference in its entirety and for all purposes as if completely and fully set forth herein.

### TECHNICAL FIELD

The present implementations relate generally to implantable devices, and more particularly to a flexible and self-bonding implantable electrostimulation device.

### BACKGROUND

Application of electrical stimulus to the nervous system of a living subject can effectively treat neurological diseases. However, conventional systems may be fixed and inflexible, and may not effectively accommodate rapid tissue growth. Conventional systems may thus impair development. For infants, children and adolescents, additional surgeries can be needed for device replacement once implanted devices are outgrown, leading to repeated interventions and complications associated with pediatric patients.

### SUMMARY

Example implementations in accordance with present implementations can adapt to in vivo nerve tissue growth with minimal mechanical constraint. An example flexible and self-bonding implantable electrostimulation device can include viscoplastic electrodes and a strain sensor, and can substantially mitigate or eliminate mechanical stress at an interface between electronics and growing tissue. The flexible and self-bonding implantable electrostimulation device can self in an aqueous in vivo environment, including during implantation surgery. Self-bonding of the flexible and self-bonding implantable electrostimulation device can advantageously allow implantation of a reconfigurable and seamless neural interface. The flexible and self-bonding implantable electrostimulation device can further advantageously accommodate growing nerves and remain implanted in vivo for months without disruption of functional behavior. The flexible and self-bonding implantable electrostimulation device in accordance with present implementations can enable growth-adaptive pediatric electronic medicine. Thus, a technological solution for a flexible and self-bonding implantable electrostimulation device is desired.

Example implementations also include an implantable device with a first substantially planar panel having a first planar surface and a second planar surface opposite to the first planar surface, and at least one electrode portion at the first planar surface, and a second substantially planar panel having a first planar surface adhered to the second planar surface of the first panel, and at least one sensor portion disposed on the first planar surface of the second panel.

Example implementations also include a device with a third substantially planar panel having a first planar surface adhered to the first planar surface of the first panel.

Example implementations also include a device where the electrode portion is at least partially enclosed between the first panel and the third panel.

Example implementations also include a device where the sensor portion is at least partially enclosed between the first panel and the second panel.

Example implementations also include a device where the electrode portion comprises a plurality of electrode portions.

Example implementations also include a device where the electrode portion includes an electrode pad portion located proximate to a first edge of the first panel.

Example implementations also include a device where the electrode portion and the sensor portion comprise poly(3,4 ethylenedioxythiophene) polystyrene sulfonate (PEDOT:PSS).

Example implementations also include a device where the electrode portion further comprises glycerol.

Example implementations also include a device where the first, second, and third panels comprise a viscoplastic polymer.

Example implementations also include a device where the first panel is bonded directly with the second panel and the third panel.

Example implementations also include a device where the one or more of the first, second and third panels are disposed around an in vivo biological structure.

Example implementations also include a device where the biological structure is a nerve, and the electrode portion is disposed at least partially in contact with the nerve.

Example implementations also include a method of manufacturing an implantable device, by depositing a component layer on at least one substrate, forming at least one electrode from the component layer, forming at least one sensor from the component layer, depositing a panel layer on the substrate, the electrode, and the sensor, adhering a cover panel including at least a first portion of the panel layer to an electrode panel including the electrode and at least a second portion of the panel layer, and adhering the electrode panel to a sensor panel including the sensor and at least a third portion of the panel layer.

Example implementations also include a method including joining the electrode with the panel layer, and joining the sensor with the panel layer.

Example implementations also include a method where the joining the electrode with the panel layer comprises joining the electrode with the panel layer by a heat treatment, and the joining the sensor with the panel layer comprises joining the sensor with the panel layer by the heat treatment.

Example implementations also include a method including separating, from the substrate, the electrode and the second portion of the panel layer to form the electrode panel, separating, from the substrate, the sensor and the third portion of the panel layer to form the sensor panel, and separating, from the substrate, the first portion of the panel layer to form the cover panel.

Example implementations also include a method of claim 13, wherein the component layer comprises poly(3,4 ethylenedioxythiophene) polystyrene sulfonate (PEDOT:PSS).

Example implementations also include a method of claim 13, wherein the panel layer comprises a viscoplastic polymer.

Example implementations also include a method of claim 13, wherein the adhering the cover panel comprises adhering the cover panel to the electrode panel in an in vivo environment and by heat of the in vivo environment, and the adhering the electrode panel comprises adhering the electrode panel to the sensor panel in the in vivo environment and by heat of the in vivo environment.

Example implementations also include an implantable device with a first substantially planar panel having a first planar surface and a second planar surface opposite to the first planar surface, at least one electrode portion at the first planar surface, and a panel elasticity corresponding to a tissue elasticity associated with an in vivo nerve, a second substantially planar panel having a first planar surface adhered to the second planar surface of the first panel, at least one sensor portion disposed on the first planar surface of the second panel, and the panel elasticity, and a third substantially planar panel having a first planar surface adhered to the first planar surface of the first panel, and the panel elasticity.

### BRIEF DESCRIPTION OF THE DRAWINGS

These and other aspects and features of the present implementations will become apparent to those ordinarily skilled in the art upon review of the following description of specific implementations in conjunction with the accompanying figures, wherein:

FIG. 1A illustrates an example flexible and self-bonding implantable electrostimulation device, in accordance with present implementations.

FIG. 1B illustrates an example flexible and self-bonding implantable electrostimulation device in an example exploded view, in accordance with present implementations.

FIG. 1C illustrates an example flexible and self-bonding implantable electrostimulation device in an example plan view, in accordance with present implementations.

FIG. 2 illustrates an example flexible and self-bonding implantable electrostimulation device affixed to an example in vivo nerve structure, in accordance with present implementations.

FIG. 3 illustrates an example voltage response waveform of an example flexible and self-bonding implantable electrostimulation device affixed to an example in vivo nerve structure, in accordance with present implementations.

FIG. 4 illustrates an example method of manufacturing a flexible and self-bonding implantable electrostimulation device, in accordance with present implementations.

FIG. 5 illustrates an example method of manufacturing a flexible and self-bonding implantable electrostimulation device, further to the example method of FIG. 4.

### DETAILED DESCRIPTION

The present implementations will now be described in detail with reference to the drawings, which are provided as illustrative examples of the implementations so as to enable those skilled in the art to practice the implementations and alternatives apparent to those skilled in the art. Notably, the figures and examples below are not meant to limit the scope of the present implementations to a single implementation, but other implementations are possible by way of interchange of some or all of the described or illustrated elements. Moreover, where certain elements of the present implementations can be partially or fully implemented using known components, only those portions of such known components that are necessary for an understanding of the present implementations will be described, and detailed descriptions of other portions of such known components will be omitted so as not to obscure the present implementations. Implementations described as being implemented in software should not be limited thereto, but can include implementations implemented in hardware, or combinations of software and hardware, and vice-versa, as will be apparent to those skilled in the art, unless otherwise specified herein. In the present specification, an implementation showing a singular component should not be considered limiting; rather, the present disclosure is intended to encompass other implementations including a plurality of the same component, and vice-versa, unless explicitly stated otherwise herein. Moreover, applicants do not intend for any term in the specification or claims to be ascribed an uncommon or special meaning unless explicitly set forth as such. Further, the present implementations encompass present and future known equivalents to the known components referred to herein by way of illustration.

Bioelectronic devices performing actions including but not limited to vagus nerve stimulation and deep brain stimulation, can be appropriate for treatment of various diseases. Elastic bioelectronics in accordance with present implementations can accommodate repeated strain induced by the dynamics of organ and body movement, and can adapt to developmental tissue growth without asserting substantial stress during the process. For example, implantable vagus nerve stimulators in accordance with present implementations, can be highly effective in reducing seizure occurrence in some patients with drug-resistant epilepsy, and can be applied in pediatric contexts for young children with tissue restriction-related issues.

A flexible and self-bonding implantable electrostimulation device in accordance with present implementations thus can advantageously have growth-adaptive properties associated with its particular structure and composition. Advantages of present implementations include a strain-rate dependent mechanical response, responsive to permanent deformation induced only by slow tissue growth, rather than fast body movement. Advantages of present implementations also include adaptation to growing tissue and changes of shape thereof, while exerting minimal stress on the interfaced tissue during growth. Advantages of present implementations also include flexibility allowing customization to particular shapes and sizes of in vivo tissues, organs, other biological structures, and the like. As one example, present implementations can accommodate large variation in organ size from person to person, with reconfigurable electronics that can be adjusted to arbitrary shapes during surgery. In addition, present implementations can actively self-bond by viscoplastic panels including electrostimulation components, and can undergo unrecoverable deformations to fit and adapt to growing tissue structures after implantation.

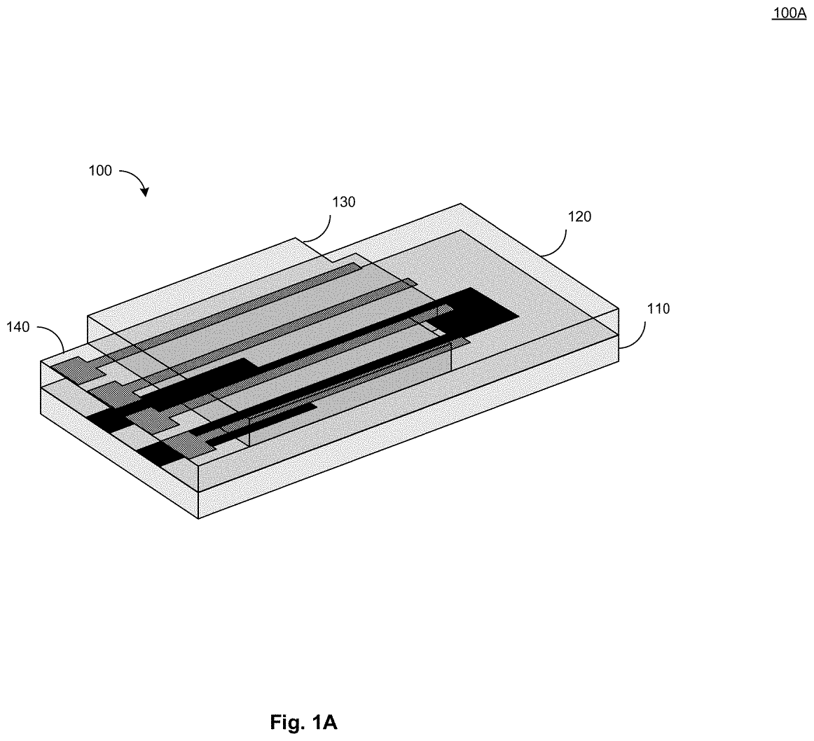

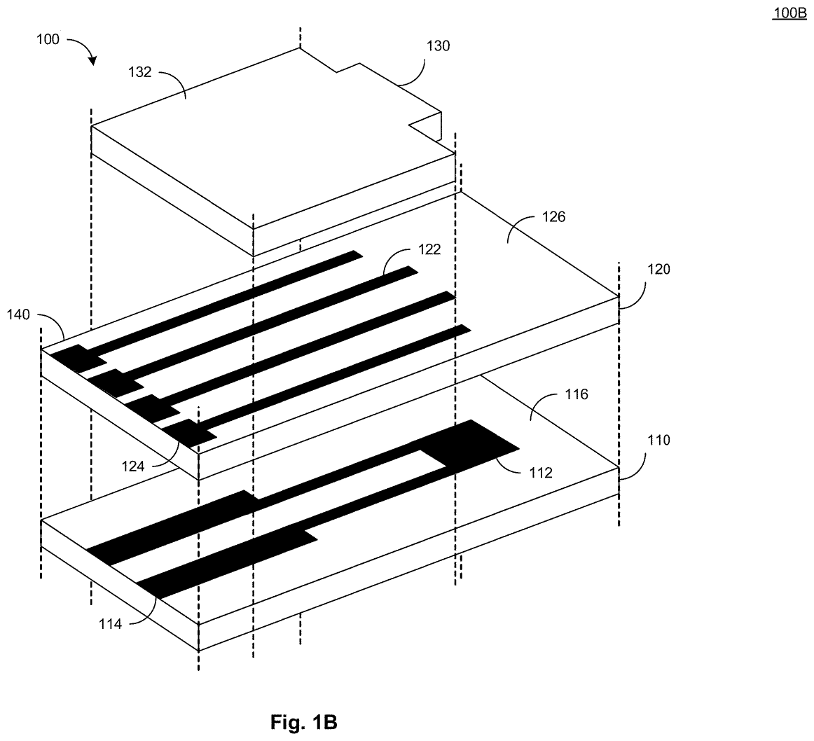

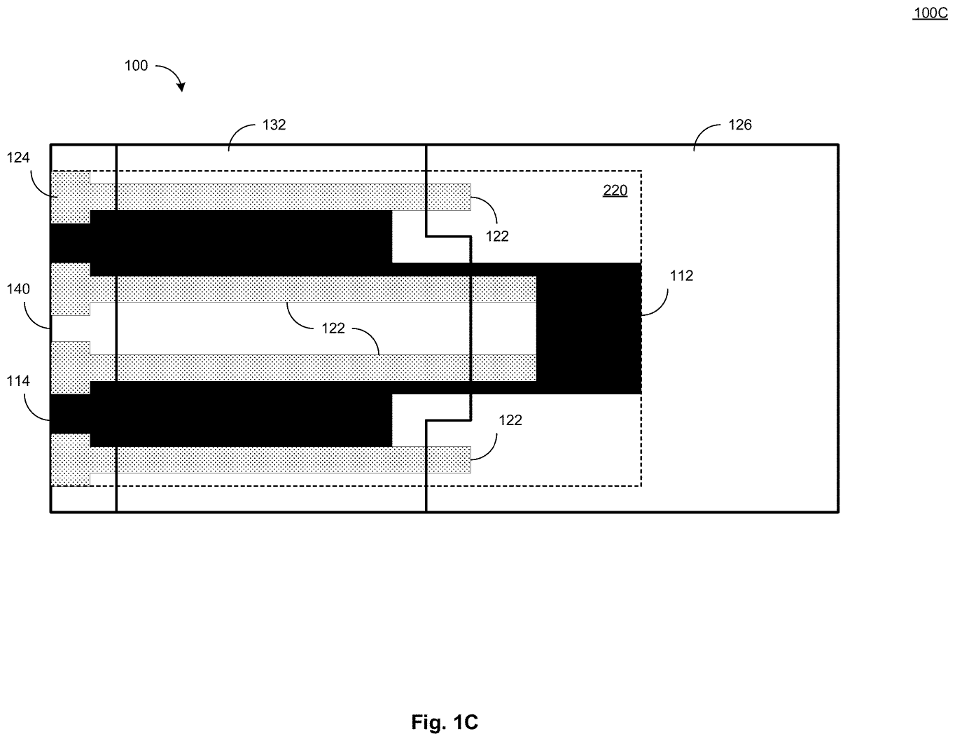

FIG. 1A illustrates an example flexible and self-bonding implantable electrostimulation device, in accordance with present implementations. As illustrated by way of example in FIG. 1A, an example implantable electrostimulation device 100 in an example assembled view 100A includes a sensor panel 110, an electrode panel 120, a cover panel 130, and an electrode interface region 140. In some implementations, a total thickness of the example implantable electrostimulation device 100 is approximately 120 μm. It is to be understood that the example implantable electrostimulation device 100 can be of arbitrary length and width to accommodate varying in vivo environments and structures therein, and to accommodate electrical connections thereof or therewith.

The sensor panel 110 is or includes a flexible substrate bondable with any panel having a like composition. In some implementations, the sensor panel 110 has a substantially rectangular planar shape and a thickness orthogonal to the rectangular plane. In some implementations, the sensor panel is bonded at a first face thereof to the electrode panel 120 and exposed at a second face thereof to an external environment, where the first face corresponds to a largest rectangular plane of the sensor panel 110, and where the second face is opposite to the first face. In some implementations, one or more electronic components can be fabricated on or within the sensor panel on a planar surface thereof. In some implementations, the sensor panel 110 is or includes a viscoplastic material having one or more conductive materials disposed thereon, therein, therewith, or the like. In some implementations, the sensor panel 110 has a tensile stress material property that varies and is different based on a rate of strain applied to the sensor panel 110. In some implementations, the sensor panel 110 has a ‘flowable liquid’ tensile stress material property at a relatively lower strain rate corresponding to a growth rate of living biological tissue. In some implementations, the sensor panel 110 has a ‘solid’ tensile stress material property at a relatively higher strain rate corresponding to organ movement, joint movement, muscle expansion and contraction, and the like. Thus, in some implementations, the sensor panel can resist deformation at higher strain rates, to prevent morphological alterations during implantation and post-implantation body movement, while adapting to gradual tissue expansion and growth. As one example, the sensor panel can have a Young's modulus corresponding to approximately 0.4 MPa measured at a strain rate of 50%/s.

The electrode panel 120 is or includes a flexible substrate bondable with any panel having a like composition. In some implementations, the electrode panel 120 is or includes one or more of a composition and a structure corresponding to the sensor panel 110. In some implementations, the electrode panel 120 has a shape substantially corresponding to the sensor panel 110. In some implementations, a largest rectangular plane of the electrode panel 120 corresponds in one or more of size and shape to the largest rectangular plane of the sensor panel 110. In some implementations, a first face of the electrode panel 120 corresponds in size and shape to one or more of the first face and the second face of the sensor panel 110. In some implementations, the electrode panel is bonded at a first face thereof to the sensor panel and a second face thereof to the cover panel 130, where the second face is opposite to the first face. In some implementations, the electrode panel 120 is an intermediate insulation layer between any electrical, conductive, or like components disposed on a first face thereof and any electrical, conductive, or like components in contact with a second face thereof.

The cover panel 130 is or includes a flexible substrate bondable with any panel having a like composition. In some implementations, the cover panel 130 is or includes one or more of a composition and a structure corresponding to the sensor panel 110. In some implementations, the cover panel 130 has a shape substantially corresponding to the electrode panel 120 in one or more directions, and a shape substantially smaller than the electrode panel in one or more directions. As one example, the cover panel can have a length less than the electrode panel 120 in a length direction. In some implementations, the cover panel 130 can cover at least a portion of the electrode panel 120 and any components thereunder, and can leave exposed any components of the electrode panel 120. In some implementations, the cover panel 130 is bonded at a first face thereof to the electrode panel 120 and exposed at a second face thereof to the exterior environment, where the second face is opposite to the first face. In some implementations, the cover panel 130 is an external insulation layer between any electrical, conductive, or like components disposed on the electrode layer 120 and the external environment. The electrode interface region 140 is a region of the electrode panel 120 left exposed by the cover panel 130. In some implementations, one or more components of the electrode panel 120 not in contact with the cover panel 120 or the sensor panel 110 are exposed to the exterior environment. As one example, the exterior environment can include but is not limited to an ambient environment and an in vivo environment.

FIG. 1B illustrates an example flexible and self-bonding implantable electrostimulation device in an example exploded view, in accordance with present implementations. As illustrated by way of example in FIG. 1B, an example implantable electrostimulation device 100 in an example exploded view 100B includes the sensor panel 110 having a sensor surface 116, the electrode panel 120 having an electrode surface 126, the cover panel 130 having an exterior surface 132, the electrode interface region 140, a deformation sensor 112 having a deformation portion 114, one or more electrodes 122, one or more electrode pads 124.

The deformation sensor 112 is or includes one or more electrical, conductive, or like materials disposed on a first surface of the sensor panel 110. The deformation sensor is or includes poly(3,4-ethylenedioxythiophene) polystyrene sulfonate (PEDOT:PSS). In some implementations, the deformation sensor also includes glycerol. In some implementations, a deformation sensor including glycerol advantageously demonstrates relative resistive stability. As one example, a PEDOT:PSS/glycerol conductor has an approximately 3.9× increase in resistance when stretched to approximately 100% strain, and a PEDOT:PSS conductor absent glycerol demonstrates an approximately 30× increase in resistance under approximately 8% uniaxial strain.

The deformation portion 114 is or includes one or more electrical, conductive, or like materials disposed on a first surface of the sensor panel 110 and integrated with the deformation sensor 112. In some implementations, the deformation portion 114 has one or more terminals operable to vary resistively with deformation. Thus, in some implementations, a degree of change in a resistive response of one or more of the deformation portion 114 and the deformation sensor 112 changes by deformation thereof in response to expansion of the sensor panel. In some implementations, this change in resistive response indicates a degree of expansion of the deformation sensor and the sensor panel 110, and correspondingly indicates a degree of expansion of the example implantable electrostimulation device 100.

The electrodes 122 are or include one or more electrical, conductive, or like materials disposed on a second surface of the electrode panel 120. In some implementations, the electrodes 122 are or include PEDOT:PSS/glycerol material corresponding to that of the deformation sensor 112. In some implementations, the electrodes 122 each include an electrode pad 124 at a first end thereof and an electrical device interface region at an opposite end thereof. As one example, each of the electrode pads can have a planar electrode size of 0.04 cm2. In some implementations, the electrode pads 124 are contactable with a biological structure, and the electrical device interface regions are contactable with terminal, lead, or the like of an electrical, electronic, or like device. As one example, the electrical device interface regions can be operatively coupled to a potentiostat, voltage sensor, or the like.

The sensor surface 116 corresponds to a first face of the sensor panel 110, and has the deformation sensor 112 disposed thereon. In some implementations, the sensor surface 116 is not exposed to the external environment, and is bonded to the first face of the electrode layer 120. The electrode surface 126 corresponds to a second face of the electrode panel 120, and has the electrodes 122 disposed thereon. In some implementations, the electrode surface 126 is bonded to the first face of the cover layer 130, and is partially exposed to the external environment where not bonded to the cover layer 130. In some implementations, the electrode surface 126 is exposed to the external environment at the electrode interface region 140. The exterior surface 132 corresponds to a second face of the cover panel 130, and is exposed to the external environment.

FIG. 1C illustrates an example flexible and self-bonding implantable electrostimulation device in an example plan view, in accordance with present implementations. As illustrated by way of example in FIG. 1C, an example implantable electrostimulation device 100 in an example plan view 100C includes the electrode surface 126, the exterior surface 132, the electrode interface region 140, the deformation sensor 112 having the deformation portion 114, the electrodes 122, the electrode pads 124, and a device region 220. The device region 220 includes the deformation sensor and the electrodes 122. It is to be understood that that the device region 220 can be a region of the example implantable electrostimulation device 100, and is not required to be a distinct physical structure. It is to be understood that the shapes of the cover panel 130 and the exterior surface 132 can vary. It is to be further understood that that relative lengths of the electrodes 112 can vary. As one example, electrodes 122 closer to edges of the electrode panel 120 can have lengths less than lengths of electrodes 122 closer to a center or interior portion of the electrode panel 122. As another example, the shape of the cover panel 130 can include notches, recesses, or the like corresponding to lengths of the electrodes 122 and providing exposure for electrical device interface regions of the electrodes 122.

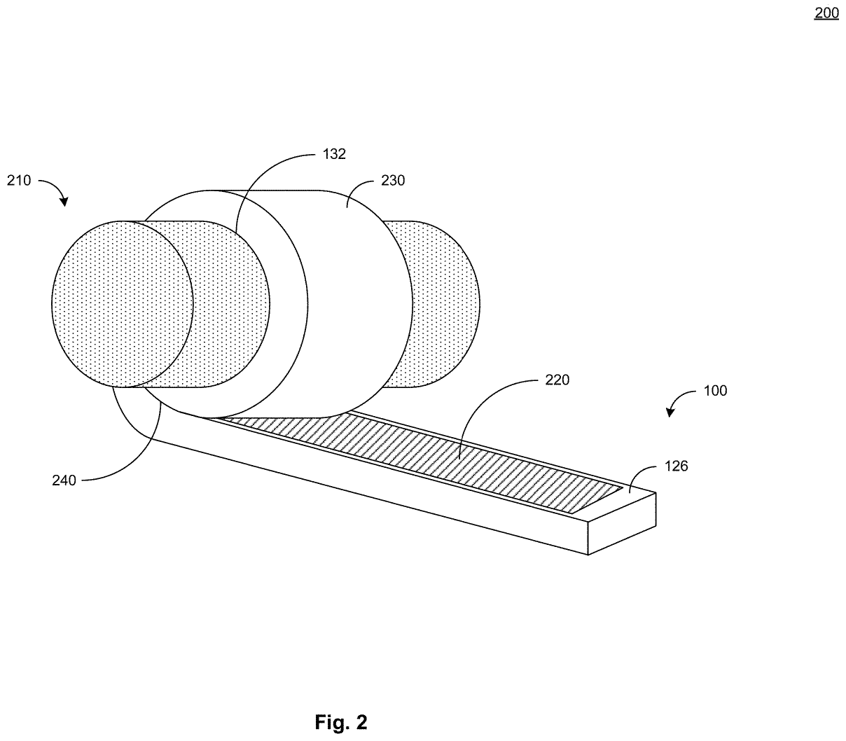

FIG. 2 illustrates an example flexible and self-bonding implantable electrostimulation device affixed to an example in vivo nerve structure, in accordance with present implementations. As illustrated by way of example in FIG. 2, an example implantable electrostimulation device 100 is affixed to an example in vivo nerve structure 210 in example view 200, and the device includes the electrode surface 126, the device region 220, the exterior surface 132, a rear exterior surface 230, and a self-bonding surface 240.

The in vivo nerve structure 210 is contactable with the example implantable electrostimulation device 100. In some implementations, the device region 220 is contactable with the in vivo nerve structure. In some implementations, the in vivo nerve structure is a nerve of a human, other mammal, or other living biological organism. As another example, the in vivo nerve structure 210 can be a sciatic nerve of a rapidly growing mammal. It is to be understood that due to the flexibility and responsiveness to strain of the example implantable electrostimulation device 100, the example implantable electrostimulation device 100 advantageously does not significantly affect or impede mechanical properties of biological tissue under strain, including the growing in vivo nerve structure 210. The rear exterior surface 230 corresponds to a second face of the sensor panel 110, and is exposed to the external environment. It is to be understood that a Young's modulus of the example implantable electrostimulation device 100 corresponding to a Young's modulus of the in vivo nerve structure prevents significant Young's modulus mismatch therebetween and stress at the interface therebetween that can deteriorate nerve functionality.

The self-bonding surface 240 corresponds to a portion of the electrode surface 126 in contact with the rear exterior surface 230. The example implantable electrostimulation device 100 can be wrapped around the nerve and subsequently attached to itself by the self-bonding surface 240, forming a soft enclosure around the in vivo nerve structure 210. The self-bonding surface can bond with the rear exterior surface 230 in an aqueous environment due to the hydrophobic nature of the PDMS backbone in PDMS-IU, and increased enthalpy gained by strong hydrogen bonding formation. Thus, in some implementations, the example implantable electrostimulation device 100 can advantageously adapt to tissue growth and maintain stable strain sensing and neuromodulation in growing organisms. As one example, the self-bonding surface can bond with the rear exterior surface 230 after approximately 5 min of contact in the in vivo environment. It is to be understood that the self-bonding surface 240 is not limited to a bonding time of 5 minutes. In some implementations, pulling of the example implantable electrostimulation device 100 after self-bonding causes no visible delamination or dislocation, resulting in a durable nerve interface capable of withstanding physiological movements.

In some implementations, the example implantable electrostimulation device 100 demonstrates particular plasticity at body temperature of approximately 37° C. As one example, the degree of plasticity of the example implantable electrostimulation device 100 can be approximately 97.2%, which is advantageously higher than conventional systems having plasticity as low as 2.4%. A degree of plasticity P can be determined by Equation (1):

$$P = \frac{{ɛ}_{t}}{{ɛ}_{max}} \qquad Eqn . ( 1 )$$

where εi is the irreversible strain after recovery and εmax is the maximum strain. Thus, in some implementations, the example implantable electrostimulation device 100 advantageously demonstrates high biocompatibility, high viscoplasticity and close-to-zero stress when the electronic material is subject to a slow strain rate. Concurrently, under fast strain rate, the example implantable electrostimulation device 100 demonstrates elastic properties and allows intimate contact between the electrode and nerve. Thus, example implantable electrostimulation device 100 can be biomechanically compatible, suture-free, and individually reconfigurable for stable and implantation to soft sciatic nerve.

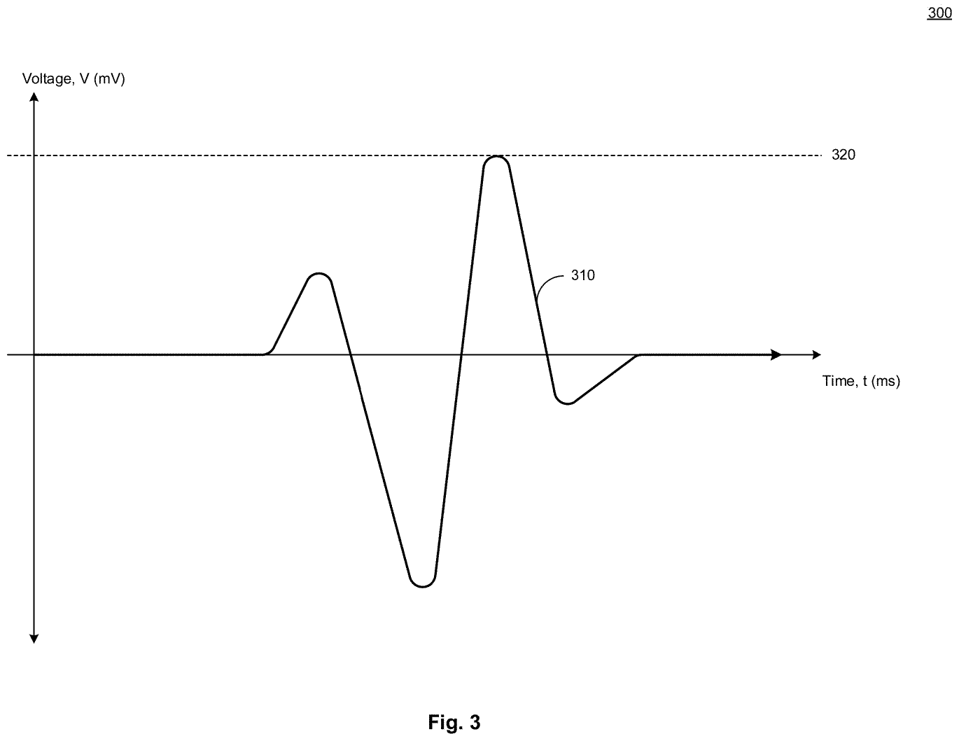

FIG. 3 illustrates an example voltage response waveform diagram of an example flexible and self-bonding implantable electrostimulation device affixed to an example in vivo nerve structure, in accordance with present implementations. As illustrated by way of example in FIG. 3, an example voltage response waveform diagram 300 includes voltage response waveform 310 and a voltage response peak level 320.

The voltage response waveform 310 is generated in response to an application of a stimulation voltage to one or more electrodes 122 of the example implantable electrostimulation device 100. In some implementations, the dual conduction of electrons and ions in PEDOT:PSS/glycerol panels of the example implantable electrostimulation device 100 and the porous interconnect associated therewith, a low threshold voltage is sufficient to induce compound action potential (CAP). As one example, a low threshold voltage can be approximately 100 mV. As one example, a 50 mV input can cause an approximately 10 μV peak output. As another example, a 100 mV input can cause an approximately 250 μV peak output. As another example, a 300 mV input can cause an approximately 1 mV peak output. As another example, a 500 mV input can cause an approximately 2 mV peak output. It is to be understood that peak output of the example implantable electrostimulation device 100 can be greater than conventional systems by at least one order of magnitude.

In some implementations, the example implantable electrostimulation device 100 has a high cathode charge storage capacity (CSCc), a strain-insensitive impedance, and maintains a stable resistance under repeated stretching and releasing stresses. It is to be understood that a higher CSCc supports higher charge injection at a given input stimulation voltage. As one example, CSCc can stabilize at approximately 137.0±7.7 mC/cm2. This level can be advantageously higher than corresponding CSCc of conventional materials including cracked Au and electrochemically deposited PEDOT. As another example, impedance of the panels 110, 120 and 130 of the example implantable electrostimulation device 100 can be approximately 26 MOhm, and the impedance of the electrodes can be 6.3 kOhm, advantageously providing a low leakage current through and high insulation capacity of the panels 110, 120 and 130.

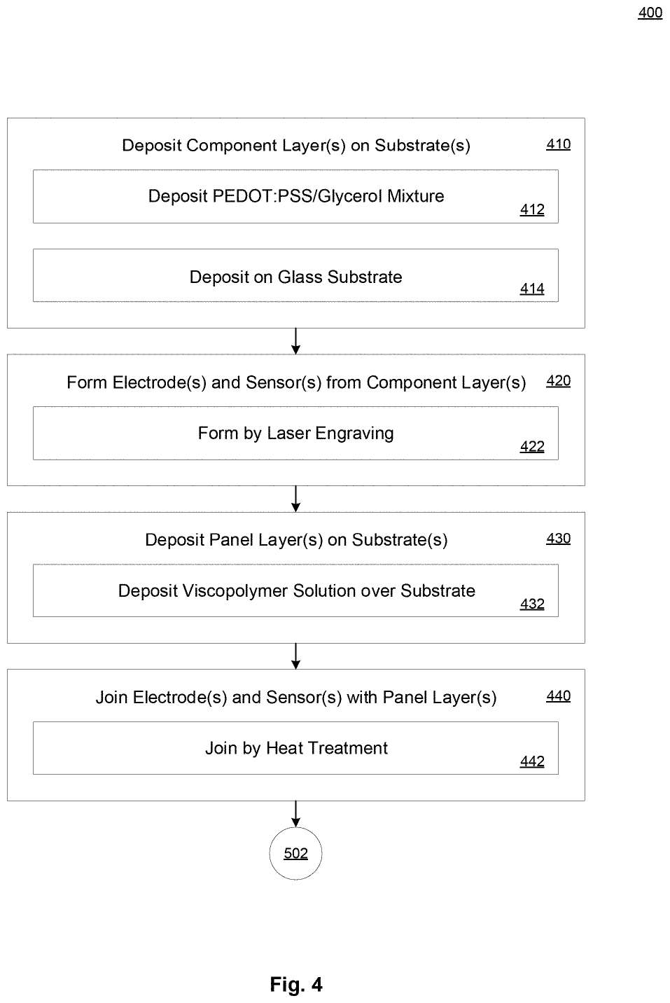

FIG. 4 illustrates an example method of manufacturing a flexible and self-bonding implantable electrostimulation device, in accordance with present implementations. In some implementations, an example system manufactures the example device 100 by method 400 according to present implementations. In some implementations, the method 400 begins at step 410.

At step 410, the example system deposits at least one component layer on at least one substrate. In some implementations, the thickness of PEDOT:PSS/glycerol is 2 μm. It is to be understood that the example system can deposit component layers on corresponding substrates, and that the number of substrates can be less than or equal to the number of composite layers. It is to be understood that that the component layer can be or include any combination of a conducting polymer and viscous additive, or any one or more materials having one or more corresponding characteristics thereto. In some implementations, step 410 includes at least one of steps 412 and 414. At step 412, the example system deposits a poly(3,4-ethylenedioxythiophene) polystyrene sulfonate (PEDOT:PSS) solution. At step 414, the example system deposits the component layer on a glass substrate. As one example, the component layer can be spin-coated on a glass substrate at a speed of 400 rpm for 1 minute. As another example, the example system can drop-cast a PEDOT:PSS/glycerol aqueous mixture on the substrate. The method 400 then continues to step 420.

At step 420, the example system forms one or more electrodes and at least one sensor from the component layer. In some implementations, step 420 includes step 422. At step 422, the example system forms at least one of the electrodes and the sensor by laser engraving. In some implementations, a feature size of conducting PEDOT:PSS electrodes of approximately 150-μm is achieved by the laser engraving process. The method 400 then continues to step 430.

At step 430, the example system deposits at least one panel layer on the substrate. In some implementations, step 430 includes step 432. At step 432, the example system deposits a viscopolymer solution on or over the substrate. As one example, viscoplastic polymers having 20 wt % in toluene can be drop casted on the PEDOT:PSS/glycerol pattern and dried in ambient conditions. The example system can vary self-bonding and viscoplastic properties by modifying the ratio between the weak dynamic bonding isophorone bisurea (IU) units and strong hydrogen bonding 4,4′-methylenebis(phenyl urea) (MPU) units in viscoplastic. In some implementations, the example system modifies a ratio of PDMS-IU (PDMS:poly(dimethylsiloxane)) and PDMS-IU0.6-MPU0.4. As one example, modulating a ratio of the IU and MPU to 7:3 resulted in a material property causing an irreversible plastic deformation at 100% uniaxial strain. As another example, a PEDOT:PSS/Glycerol with an example viscoplastic demonstrates viscoelastic behavior at strain rates higher than 5%/s and zero stress at a lower strain rate of 0.05%/s, substantially eliminating mechanical constraint on and interference in growth of an in vivo nerve at a normal growth rate. As one example, a normal growth rate can be approximately 2×10−5%/s. Thus, in some implementations, the viscoplastic is or includes a polymer blend of PDMS-IU and PDMS-IU0.6-MPU0.4 as a viscoplastic insulator. The method 400 then continues to step 440.

At step 440, the example system joins the electrodes and the sensor with a corresponding one or portion of the at least one panel layer. In some implementations, step 440 includes step 442. At step 442, the example system joins the electrodes and the sensor with a corresponding one or portion of the at least one panel layer by a heat treatment. As one example, the example system can apply heat at 70° C. for 2 hours to facilitate joining of a viscoplastic panel with PEDOT:PSS/glycerol electrodes or sensor. As another example, the joining of a viscoplastic panel with PEDOT:PSS/glycerol electrodes or sensor can occur, or can also occur, in an ambient environment of approximately 25° C., with or without application of further or additional heat. The method 400 then continues to step 502.

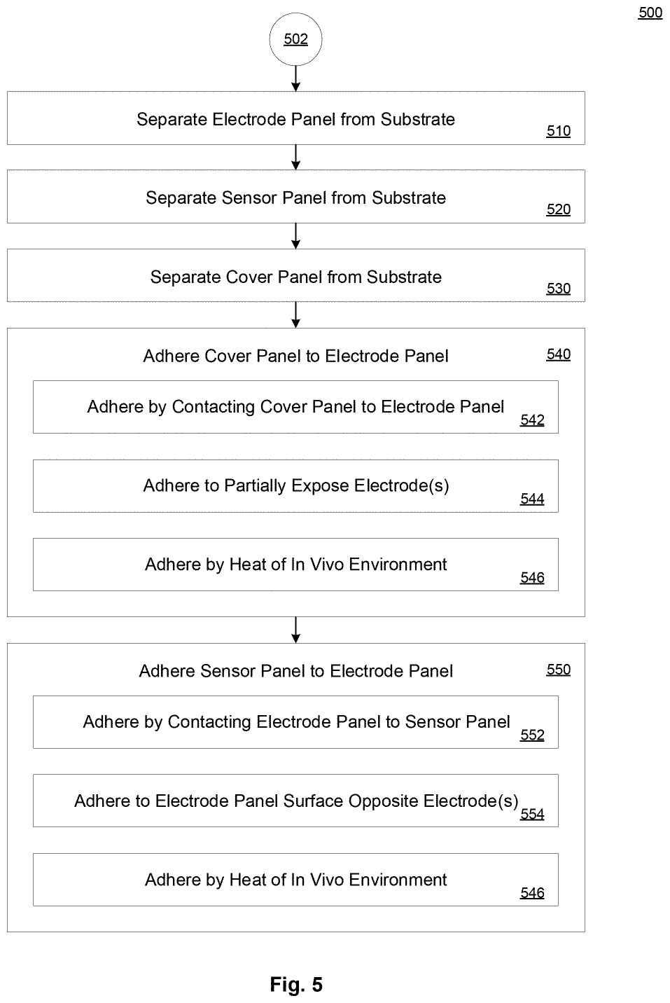

FIG. 5 illustrates an example method of manufacturing a flexible and self-bonding implantable electrostimulation device, further to the example method of FIG. 4. In some implementations, an example system manufactures the example device 100 by method 500 according to present implementations. It is to be understood that a self-bonding mechanism is especially advantageous for surgical procedures because it allows in situ device reconfiguring and reshaping during implantation, and thus enables adequately customized fitting without prior information about the morphology and size of the in vivo biological structures to which an example implantable electrostimulation device 100 can be affixed. In some implementations, the method 500 begins at step 502. The method 500 then continues to step 510.

At step 510, the example system separates an electrode panel from the substrate. The method 500 then continues to step 520. At step 520, the example system separates a sensor panel from the substrate. The method 500 then continues to step 530. At step 530, the example system separates a cover panel from the substrate. The method 500 then continues to step 540.

At step 540, the example system adheres the cover panel to the electrode panel. In some implementations, step 540 includes at least one of steps 542, 544 and 546. At step 542, the example system adheres the cover panel to the electrode panel by contacting the cover panel to the electrode panel. At step 544, the example system adheres the cover panel to the electrode panel to at least partially expose one or more portions of the electrodes. In some implementations, the example system can expose one or more corresponding electrode pads 124 of the electrodes 122. At step 546, the example system adheres the cover panel to the electrode panel by heat of an in vivo environment. The method 500 then continues to step 550.

At step 550, the example system adheres the sensor panel to the electrode panel. In some implementations, step 550 includes at least one of steps 552, 554 and 556. At step 552, the example system adheres the sensor panel to the electrode panel by contacting the sensor panel to the electrode panel. At step 554, the example system adheres the sensor panel to a surface of the electrode panel opposite to a surface including the electrodes. At step 556, an in vivo environment adheres the sensor panel to the electrode panel by heat of an in vivo environment. As one example, the example implantable electrostimulation device 100 self-bonds subsequent to in vivo implantation by the body heat of the organism at the in vivo implantation site. As another example, self-bonding can occur, or can also occur, in an ambient environment of approximately 25° C., with or without application of further or additional heat. In some implementations, the method 500 ends at step 550.

The herein described subject matter sometimes illustrates different components contained within, or connected with, different other components. It is to be understood that such depicted architectures are illustrative, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Further, unless otherwise noted, the use of the words “approximate,” “about,” “around,” “substantially,” etc., mean plus or minus ten percent.

The foregoing description of illustrative implementations has been presented for purposes of illustration and of description. It is not intended to be exhaustive or limiting with respect to the precise form disclosed, and modifications and variations are possible in light of the above teachings or may be acquired from practice of the disclosed implementations. It is intended that the scope of the invention be defined by the claims appended hereto and their equivalents.

What is claimed is:

1. An implantable medical device capable of self-transforming shape and morphology thereof responsive to mechanical force caused by growth expansion of a biological structure, to accommodate in vivo tissue growth, the implantable medical device comprising:
a first substantially planar panel having a first planar surface and a second planar surface opposite to the first planar surface, and at least one electrode portion at the first planar surface; and
a second substantially planar panel having a first planar surface adhered to the second planar surface of the first panel, and at least one sensor portion disposed on the first planar surface of the second panel.

2. The device of claim 1, further comprising:
a third substantially planar panel having a first planar surface adhered to the first planar surface of the first panel.

3. The device of claim 2, wherein the electrode portion is at least partially enclosed between the first panel and the third panel.

4. The device of claim 1, wherein the sensor portion is at least partially enclosed between the first panel and the second panel.

5. The device of claim 1, wherein the electrode portion comprises a plurality of electrode portions.

6. The device of claim 1, wherein the electrode portion includes an electrode pad portion located proximate to a first edge of the first panel.

7. The device of claim 1, wherein the electrode portion and the sensor portion comprise poly(3,4-ethylenedioxythiophene) polystyrene sulfonate (PEDOT:PSS).

8. The device of claim 1, wherein the electrode portion further comprises glycerol.

9. The device of claim 1, wherein the first, second, and third panels comprise a viscoplastic polymer, that resists deformation by fast movement, and is deformable and mechanically responsive to slow tissue growth.

10. The device of claim 1, wherein the first panel is bonded directly with the second panel and the third panel.

11. The device of claim 1, wherein the one or more of the first, second and third panels are disposed around an in vivo biological structure.

12. The device of claim 11, wherein the biological structure is a nerve, and the electrode portion is disposed at least partially in contact with the nerve.

13. A method of manufacturing an implantable medical device capable of self-transforming shape and morphology thereof responsive to mechanical force caused by growth expansion of a biological structure, to accommodate in vivo tissue growth, the implantable medical device comprising:
depositing a component layer on at least one substrate;
forming at least one electrode from the component layer;
forming at least one sensor from the component layer;
depositing a panel layer on the substrate, the electrode, and the sensor;
adhering a cover panel including at least a first portion of the panel layer to an electrode panel including the electrode and at least a second portion of the panel layer; and
adhering the electrode panel to a sensor panel including the sensor and at least a third portion of the panel layer.

14. The method of claim 13, further comprising:
joining the electrode with the panel layer; and
joining the sensor with the panel layer.

15. The method of claim 14, wherein the joining the electrode with the panel layer comprises joining the electrode with the panel layer by a heat treatment, and the joining the sensor with the panel layer comprises joining the sensor with the panel layer by the heat treatment.

16. The method of claim 13, further comprising:
separating, from the substrate, the electrode and the second portion of the panel layer to form the electrode panel;
separating, from the substrate, the sensor and the third portion of the panel layer to form the sensor panel; and
separating, from the substrate, the first portion of the panel layer to form the cover panel.

17. The method of claim 13, wherein the component layer comprises poly(3,4-ethylenedioxythiophene) polystyrene sulfonate (PEDOT:PSS).

18. The method of claim 13, wherein the panel layer comprises a viscoplastic polymer, that resists deformation by fast movement and is deformable, and mechanically responsive to slow tissue growth.

19. The method of claim 13, wherein the adhering the cover panel comprises adhering the cover panel to the electrode panel in an in vivo environment and by heat of the in vivo environment, and the adhering the electrode panel comprises adhering the electrode panel to the sensor panel in the in vivo environment and by heat of the in vivo environment.

20. An implantable medical device capable of self-transforming shape and morphology thereof responsive to mechanical force caused by growth expansion of a biological structure, to accommodate in vivo tissue growth, the implantable medical device comprising:
a first substantially planar panel having a first planar surface and a second planar surface opposite to the first planar surface, at least one electrode portion at the first planar surface, and a panel elasticity corresponding to a tissue elasticity associated with an in vivo nerve;
a second substantially planar panel having a first planar surface adhered to the second planar surface of the first panel, at least one sensor portion disposed on the first planar surface of the second panel, and the panel elasticity; and
a third substantially planar panel having a first planar surface adhered to the first planar surface of the first panel, and the panel elasticity.
